(19) United States
(12) Patent Application Publication
(10) Pub. No.: US 2025/0177030 A1
(43) Pub. Date: Jun. 5, 2025

(54) Device, Method and System for the Irreversible Electroporation of Tissue

(71) Applicant: Stockert GmbH, Freiburg im Breisgau (DE)

(72) Inventors: Fabian Kees, Freiburg (DE); Thomas De Freitas, Freiburg (DE); David Stork, Freiburg (DE)

(21) Appl. No.: 18/952,211

(22) Filed: Nov. 19, 2024

(30) Foreign Application Priority Data
Nov. 30, 2023 (DE) 10 2023 133 549.9

(51) Int. Cl. A61B 18/12; A61B 18/00; A61B 18/14

(52) U.S. Cl. CPC A61B 18/1206; A61B 18/1492; A61B 2018/00613; A61B 2018/00702; A61B 2018/00875; A61B 2018/1253; A61B 2018/126

(57) ABSTRACT

The present invention describes a device, systems and a method which effect irreversible electroporation by means of energy-monitored control. An exemplary embodiment of the device has an electrical signal generator, an electrode pair, and an evaluation and control unit connected to the signal generator.

### CROSS-REFERENCE TO RELATED APPLICATIONS

The present application claims the benefit of German patent application DE 10 2023 133 549.9 filed on Nov. 30, 2023, the contents of which are incorporated by reference herein in their entirety.

### BACKGROUND AND SUMMARY

A device, a method and a system for the irreversible electroporation of tissue are presented here. Features and properties of the device, of the system and of the method are defined in the claims; however, the description and the figures also disclose characteristics of the device, of the system and of the method as well as of the various aspects and relationships thereof.

The treatment of tissue by means of pulsed electrical fields has gradually gained importance as a clinical application in recent years. The effects of the short high-voltage pulses used in such treatment and of the resulting high electrical field strengths on/in tissue, on the other hand, have been part of various research projects for over four decades. Such an application can be classified as a non-thermal procedure and is based on the delivery of short high pulses to/in tissue in order to generate a locally high electrical field, which can typically lie in the region of several hundred volts per centimetre. Pores are thereby generated in the cell membranes of the tissue. If this electrical field exceeds a certain threshold value during pore formation at the lipid bilayers of the cell membrane, the so-called electroporation can be irreversible and the pores remain permanently open, which ultimately leads to apoptosis (programmed cell death) of the cell.

Irreversible electroporation (IRE) is a predominantly non-thermal procedure which effects an increase in the tissue temperature by not more than a few degrees for a few milliseconds. This distinguishes IRE from RF ablation (high-frequency/−radiofrequency ablation) which is conventionally used and in which the tissue temperature increases by 20 to 70° C. and the cells are destroyed by heating. In IRE, bipolar pulses are typically used, that is to say combinations of positive and negative pulses, in order to avoid as far as possible muscle contractions, which typically occur in the case of the application of DC voltage. The pulses can be applied, for example, between two bipolar electrodes of a catheter or between a catheter electrode and a body surface electrode, which is typically stuck to the skin of the patient's back.

In order that the IRE pulses generate the desired pores in the cell membranes of the target tissue, the electrical field strength E, defined by the pulses, at/in the target tissue between a pair of at least two electrodes must exceed a tissue-dependent threshold value Eth. For example, the threshold value for cardiac cells is approximately 500 V/cm, while for bones it is 3000 V/cm. These differences in the threshold values of the field strengths allow the selective application of IRE in different tissues or mixed tissues (adipose tissue, myocardial tissue and nerve tissue). In order to achieve the required field strength, the voltage to be applied to an electrode pair depends both on the nature of the target tissue and also on the distance between the electrodes and on the size of the electrodes themselves. These parameters likewise also influence the thermal energy input during the ablation and thus the temperature peaks which can occur at the tissue to be treated. The applied voltages can reach up to 2000 V, which is substantially higher than the voltages of 10-200 V that are typical in the case of thermal RF ablation.

The bipolar pulsed field ablation pulse (the bipolar PFA pulse) for IRE comprises a positive and a negative pulse, which are applied between two electrodes with a pulse width of from 1 to 5 us and an interval between the positive and negative pulses of from 1 to 5 us. The bipolar pulses are combined to form pulse sequences, wherein each sequence can comprise over one hundred bipolar pulses with a pulse-to-pulse interval of from 1 to 10 ms. The pulse sequences in each case form a burst, wherein the entire pulse packet of the IRE ablation is composed of from 1 to 20 bursts/burst units, each of which has a burst-to-burst interval of from 1 to 1000 ms. The total duration of an ablation can be up to 10 s.

The described parameters of the pulse protocol should be set prior to the ablation such that the desired electroporation effect and an associated clinical efficacy are achieved and at the same time possible risks such as, for example, muscle contraction or thermal damage to the tissue are avoided. In addition to the temporal and quantitative variables of the pulse protocol, the electrical parameters are also of critical importance here.

Hitherto, the electrical parameters have been set in comparison systems by the specification of a target current which flows between at least two electrodes through the tissue to be treated and thus locally induces an electrical field. The size of this induced electrical field is dependent on the electrical impedance of the tissue. However, the tissue impedance likewise fluctuates according to the position or size of the electrodes as well as in dependence on the patient and thus ultimately also on the energy that is transmitted by the procedure into the tissue and can locally heat the tissue. Thus, regulating the target current in this manner does not ensure that locally undesirable thermal effects cannot occur, such as, for example, the formation of blisters at the electrodes or charring of tissue.

A further important factor for controlling the PFA pulses is the dependence of the electrical tissue impedance on the applied electrical field strength E, since this directly influences the conductivity of the tissue and thus also the local energy input. Accordingly, if the measurement of the electrical tissue impedance is made at a different electrical field strength than the actual IRE ablation, the actual energy input into the tissue during the ablation is consequently also different.

Some comparison systems use active regulation during the ablation in order to regulate the energy input, but it is not ensured that thermal damage has not already occurred, for example during the adjustment at the start of the ablation.

WO 2022/164750 A1 discloses voltage-controlled pulse sequences for IRE systems. A disclosed system has an ablation catheter with a catheter electrode. The catheter electrode generates an electrical field in the target tissue. An additional controller is configured to receive a first pulse voltage of a first pulse sequence and to determine a charge voltage on the basis of the first pulse voltage. An additional generator is configured to deliver a second pulse sequence at a controlled pulse voltage.

US 2021/0228260 A1 discloses a system and a method for a customisable waveform and control for pulsed electrical field ablation. The method discloses inter alia the following steps: configuring a first output therapy parameter set using a selected therapy profile; generating one or more first therapy outputs using the output therapy parameter set; sensing one or more first feedback parameters; comparing the first feedback parameters with an expected feedback parameter in order to generate one or more first comparison results, wherein the expected feedback parameter is associated with the selected therapy profile; and configuring a second output therapy parameter set using the first comparison results.

EP 3 964 153 A1 discloses a method for impedance-based irreversible electroporation. The method comprises measuring a tissue impedance and calculating an impedance threshold value. The impedance threshold value which is inherent in each selected protocol is calculated on the basis of protocol parameters, or read from a predetermined reference, for example from an empirical reference or precalculated references which are stored in a look-up table. In order to achieve the optimal energy for the ablation process, the protocol, more specifically the pulse duration and/or the number of pulses and/or the number of bursts, is adjusted on the basis of the measured tissue impedance. The values of the voltage peaks are typically not reduced when the protocol is adjusted.

U.S. Pat. No. 2,011,238 056 A1 discloses a system and a method for the impedance-mediated control of power delivery for electrosurgery. The system and the method disclose a series of pulses with an initial pulse, the profile of which corresponds to a preset radiofrequency starting value. Starting with the radiofrequency starting value, a radiofrequency level increases at a ramping rate to a preset radiofrequency value.

There are further known documents EP 3 232 967 A1, US 2022/0313346 A1, US 2007/0078453 A1, WO 2022/173875 A1, WO 2022/258034 A1, WO 2020/097276 A1.

In view of the mentioned prior art, the problem consists further in controlling the delivery of energy into the tissue to be treated prior to an IRE procedure. In particular, it should not be necessary to have to implement a control loop during the energy delivery in order to avoid any undesirable thermal effects, for example during adjustment.

In order to solve this problem a device according to claim 1, a method according to claim 7, and systems according to claims 4, 5 and 6 are proposed.

According to a first aspect, a device for the tissue-type-selective irreversible electroporation of a tissue is proposed. The device has an electrical signal generator.

The electrical signal generator is adapted to generate and transmit an electrical signal in accordance with a signal protocol to be received. The device has an electrode pair, for example precisely one single electrode pair. The electrode pair is (electrically) connected to the electrical signal generator. The electrode pair is adapted to receive the electrical signal. The electrode pair is adapted to close an electrical connection via the tissue lying between the electrode pair. The device has an evaluation and control unit. The evaluation and control unit is (electrically) connected to the signal generator. The evaluation and control unit is adapted, in particular in a first operating phase, to transmit a measuring signal protocol to the signal generator. The evaluation and control unit is adapted, in particular in a first operating phase, to receive at least one measuring signal transmitted by the tissue. The evaluation and control unit is adapted, in particular in a first operating phase, to determine a tissue impedance, for example precisely one single tissue impedance, on the basis of the at least one received measuring signal. The evaluation and control unit is adapted, in particular in a second operating phase, to adjust at least one format (e.g. from a large number of formats) of a burst-signal sequence protocol (predefined by a user) on the basis of the determined tissue impedance in order (by means of the adjusted burst-signal sequence protocol) to specify/define (for the signal generator) an amount of energy per burst (in joules).

This has the advantage that fluctuations in the amount of energy per burst, which are caused by tissue impedance and/or electrode geometry, are compensated for prior to the actual ablation (application of a burst-signal sequence to the tissue).

The transmitted measuring signal can have a measuring signal level, in particular first voltage level, which is equal to a burst-signal sequence level, in particular a second voltage level.

This has the advantage that the tissue impedance is explicitly measured with the same applied voltage with which the following ablation is also to be carried out. Because the impedance is dependent on the applied voltage, it is ensured in this manner that the impedance that is actually present in the later ablation is ascertained, and the formats can be adjusted on the basis thereof.

The electrode pair can have precisely one single electrode pair or can be configured as precisely one single electrode pair. The tissue impedance can have precisely one single tissue impedance or can be configured as precisely one single tissue impedance.

The signal generator can be configured as a voltage source, in particular as a high-voltage signal generator. The signal generator can be adapted to deliver high-voltage direct current (DC) pulsed field ablation (PFA) pulses.

The evaluation and control unit can be adapted, in particular in a first operating phase, to measure an electric current and an electrical voltage at least once/at least twice/many times and to determine therefrom at least one/at least two/a large number (of) (average) tissue impedance(s).

The evaluation and control unit can be adapted, in particular in a second operating phase, to adjust at least one combination of (at least two) formats (of a large number of formats) of a burst-signal sequence protocol on the basis of the determined tissue impedance in order to specify/define an amount of energy per burst. The specified/defined amount of energy per burst can exceed a threshold value of an electrical field strength induced in/at the tissue that is necessary to achieve irreversible electroporation.

In other words, the evaluation and control unit can monitor/control (by means of a protocol) temporal and/or electrical parameters of the electrical signal.

The evaluation and control unit can be arranged in the signal generator.

The evaluation and control unit is adapted, in particular in a third operating phase, to transmit the adjusted burst-signal sequence protocol to the signal generator.

The specified/defined amount of energy per burst can, in particular in the third operating phase, induce an electrical field strength in/at the, in particular myocardial, tissue. The specified/defined amount of energy per burst can be greater than an electrical field strength necessary for forming, in particular irreversibly, pores in tissue.

The formats of the burst-signal sequence protocol can specify properties and/or the amount of energy per burst of a burst-signal sequence to be generated by the signal generator.

The format(s) can have: a first number of bursts within the burst-signal sequence, at least one first time interval between at least two successive bursts of the burst-signal sequence, a second number of bipolar pulses within a burst, at least one second time interval between at least two successive bipolar pulses within a burst, a third time interval between a positive and a negative pulse of at least one bipolar pulse, a pulse width of a positive and/or negative pulse of at least one bipolar pulse, a value of a pulse deflection of a positive and/or negative pulse of at least one bipolar pulse.

The evaluation and control unit can be adapted, in particular in a second operating phase, to adjust at least one (of a large number of) format(s) of a burst-signal sequence protocol (predefined by a user) on the basis of the determined tissue impedance in order to specify/define an amount of energy per burst in dependence on the first time interval.

A first number of bursts within the burst-signal sequence can lie in a value range of from 1 to 100 burst units.

At least a first time interval between two successive bursts of the burst-signal sequence can lie in a value range of from 1 ms to 1000 ms.

A second number of bipolar pulses within a burst can lie in a value range of from 1 to 300 bipolar pulse units.

At least one second time interval between at least two successive bipolar pulses within a burst can lie in a value range of from 1 to 10 ms. A third time interval between a positive and a negative pulse can lie in a value range between 1 and 5 us.

A pulse width of a positive and/or negative pulse can lie in a value range between 1 and 10 us. A pulse width of a positive pulse can be different from a pulse width of a negative pulse.

A value of a pulse deflection of a positive pulse can lie in a value range of from 200 to 2000 V. A value of a pulse deflection of a negative pulse can lie in a value range of from −200 to −2000 V.

According to a second aspect, a system for the irreversible electroporation of a tissue is proposed. The system has a device according to the first aspect and a monopolar catheter. The catheter has a distal end. The electrode pair is configured as a first electrode and a body surface electrode. The first electrode is arranged at the distal end of the catheter and the body surface electrode is arranged on a body surface of a patient. The first electrode can be arranged in a catheter and project from the catheter at the distal end. The catheter can have a shaft at the end of which the first electrode is arranged/attached.

According to a third aspect, a system for the irreversible electroporation of a tissue is proposed. The system has a device according to the first aspect for the tissue-type-selective irreversible electroporation of a tissue, and a bipolar catheter. The catheter has a distal end. The electrode pair is configured as an electrode pair that is arranged at the distal end of the bipolar catheter. The electrode pair can be arranged in the bipolar catheter and project from the bipolar catheter at the distal end.

The device for the tissue-type-selective irreversible electroporation of a tissue offers the advantage that it can be combined with one of various catheter systems. The device can use both unipolar and bipolar multi-electrode catheter systems with the same energy activation, since variations in the electrode size and in the electrode spacing between the positive and negative pole are reflected in the measured tissue impedance, and the same maximum energy can thus be ensured during the ablation irrespective of the choice of catheter.

According to a fifth aspect, a method for the irreversible electroporation of a tissue is proposed. The method comprises providing an electrical signal generator. The signal generator is adapted to generate and transmit an electrical signal in accordance with a signal protocol to be received. The method comprises providing an electrode pair which is connected to the electrical signal generator. The electrode pair is adapted to receive the electrical signal and to close an electrical connection via the tissue lying between the electrode pair. The method comprises providing an evaluation and control unit connected to the signal generator. The method comprises transmitting, in a first operating phase, a measuring signal protocol to the signal generator by means of the evaluation and control unit. The method comprises transmitting, in the first operating phase, at least one measuring signal through the tissue by means of the signal generator and the electrode pair. The method comprises determining, in the first operating phase, a tissue impedance on the basis of the measuring signal transmitted through the tissue (and received by the electrode pair/the evaluation and control unit). The method comprises adjusting, in a second operating phase, at least one format of a burst-signal sequence protocol by means of the evaluation and control unit on the basis of the tissue impedance, in order to specify an amount of energy per burst. The method comprises transmitting, in a third operating phase, the adjusted burst-sequence protocol to the signal generator by means of the evaluation and control unit.

Further features, properties, advantages and possible modifications will become clear to a person skilled in the art from the following descriptions, in which reference is made to the accompanying drawings.

### BRIEF DESCRIPTION OF THE DRAWINGS

FIG. 1 shows a schematic representation of a bipolar pulse,

FIG. 2 shows a schematic representation of an example of an implemented pulse protocol, and

FIG. 3 shows, schematically, method steps for the irreversible electroporation of a tissue.

### DETAILED DESCRIPTION OF EMBODIMENTS

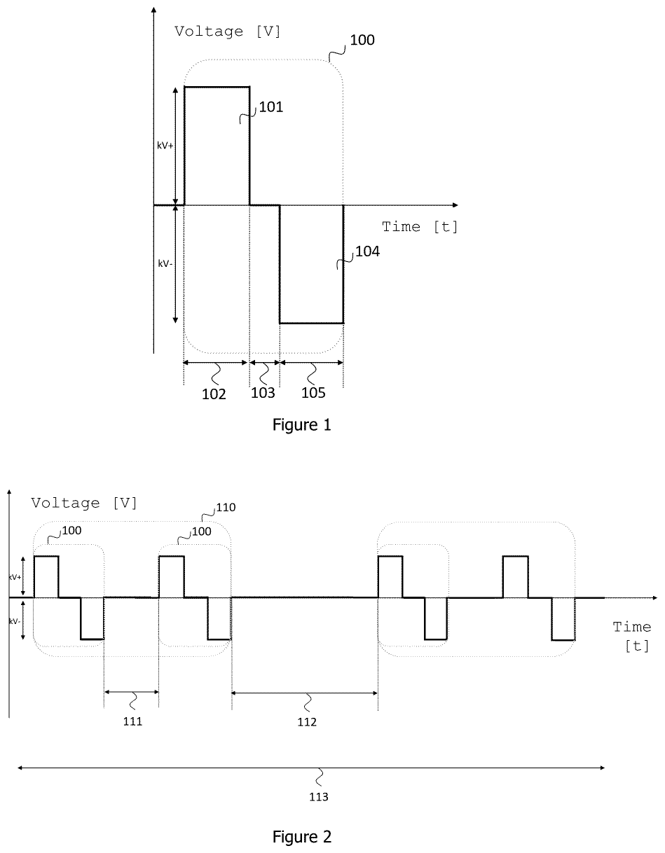

FIG. 1 shows a schematic representation of a bipolar pulse 100 which is generated by the signal generator when the evaluation and control unit transmits a burst-signal sequence protocol to the signal generator. In the present case, the signal generator is configured as a voltage source and is not shown in FIG. 1. In the present example, the formats which determine properties of the bipolar pulse 100 have been predefined by a user. In the example shown, the values of the pulse deflection kV+, kV-of the positive pulse 101 and of the negative pulse 104 are ±500 kV. The third time interval 103 between the positive pulse 101 and the negative pulse 104 is 2.5 us. The pulse width 102 of the positive pulse 101 is different from the pulse width 105 of the negative pulse 104. The difference in the pulse widths is not shown in FIG. 1.

If the tissue impedance is measured in a first operating phase, a protocol in accordance with the formats explained above is transmitted by the evaluation and control unit to the signal generator, which then implements the transmitted measuring signal protocol and transmits a measuring signal through the tissue. In the present example, the bipolar pulse 100 shown is generated and transmitted as the measuring signal. On the basis of the bipolar pulse 100 transmitted through the tissue, the evaluation and control unit determines a tissue impedance. The tissue impedance is used by the evaluation and control unit as the basis for an adjustment of the above formats.

In the present case, the tissue is myocardial tissue, and there is derived from the tissue impedance the minimum electrical field strength that must be induced in the myocardial tissue in order for irreversible electroporation to take place in the myocardial tissue.

In the present case, the third time interval 103 between the positive pulse 101 and the negative pulse 104 is reduced by the evaluation and control unit. Alternatively, the pulse widths 102, 105 could each be increased by a different amount. In other words, the pulse widths 102, 105, the third time interval 103 and the values of the pulse deflection kV+, kV-can each be configured independently of the others.

If the formats have been adjusted by the evaluation and control unit, a burst-signal sequence protocol is transmitted in a third phase of operation by the evaluation and control unit to the signal generator configured as a voltage source, which then transmits a burst-signal sequence for irreversible electroporation through the tissue.

FIG. 2 shows, schematically, this burst-signal sequence. Two bursts can be seen, one of which is provided with reference sign 110. Each burst has two bipolar pulses 100. Each bipolar pulse 100 that occurs in the burst-signal sequence has the properties which the adjusted format from the preceding figure description has newly defined/adjusted on the basis of the measured tissue impedance. The first number of bursts, here by way of example two bursts, the second time interval 111, and the first time interval 112 between two successive bursts 110 have been specified by a user before a first operating phase. The burst-signal sequence which can be seen extends over a duration 113 which corresponds to the duration of the irreversible electroporation.

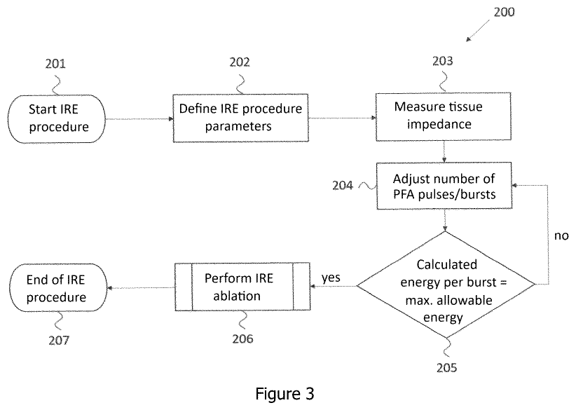

FIG. 3 shows, schematically, the steps which the method 200 for irreversible electroporation comprises. The method is referred to in the following as an irreversible electroporation procedure or IRE procedure for short. The IRE procedure starts with step 201. Then, in step 202, a user, for example a doctor, predefines the values of the individual formats. In other words, the doctor allocates a value to each format. This can be effected by manually inputting the corresponding data at the evaluation and control unit. The values of the formats and thus the property of a signal sequence to be generated have now been predefined. In a third step, the tissue impedance is measured. To this end, the evaluation and control unit transmits a measuring signal protocol to the signal generator. The measuring signal protocol indicates to the signal generator the values with which the formats which are necessary to generate a bipolar pulse are to be implemented. A measuring pulse is then transmitted through the tissue by the signal generator and received again. On the basis of this received measuring pulse, a tissue impedance is measured, from which there can be derived the electrical field strength that must be induced in the tissue in order for an IRE to take place.

For an iterative adjustment of the formats of a burst-signal sequence protocol following the tissue impedance measurement, a first number of bursts and a second number of bipolar pulses are adjusted in step 204 to the formats previously defined by the user. A calculation is then carried out 205 to determine the energy per burst which does not exceed the maximum allowable energy for the following ablation but ensures the ablation itself. In other words, at least one format or a combination of formats is adapted so that, for example, charring or water vapour formation in the tissue or neighbouring tissue is avoided on later ablation, but the IRE takes place. If this iterative adjustment is complete, the evaluation and control unit transmits the adjusted burst-signal sequence protocol to the signal generator, which then, after approval by a user, for example a doctor, performs the ablation 206. On completion of the ablation, the IRE procedure is complete 207.

1. A device for the tissue-type-selective irreversible electroporation of a tissue, comprising:
an electrical signal generator adapted to generate and transmit an electrical signal in accordance with a signal protocol to be received;
an electrode pair connected to the electrical signal generator and adapted to receive the electrical signal and to close an electrical connection via tissue lying between the electrode pair;
an evaluation and control unit connected to the signal generator and adapted:
in a first operating phase, to transmit a measuring signal protocol to the signal generator and to determine a tissue impedance on the basis of at least one measuring signal transmitted through the tissue,
in a second operating phase, to adjust at least one format of a burst-signal sequence protocol on the basis of the determined tissue impedance in order to specify an amount of energy per burst,
in a third operating phase, to transmit the adjusted burst-signal sequence protocol to the signal generator.

2. The device according to claim 1, wherein the specified amount of energy per burst, in particular in the third operating phase, induces an electrical field strength in the tissue that is greater than an electrical field strength necessary for forming, in particular irreversibly, pores in the tissue.

3. The device according to claim 1, wherein the formats of the burst-signal sequence protocol specify properties and the amount of energy per burst of a burst-signal sequence to be generated by the signal generator, comprising:
a first number of bursts within the burst-signal sequence,
at least one first time interval between at least two successive bursts of the burst-signal sequence,
a second number of bipolar pulses within a burst,
at least one second time interval between at least two successive bipolar pulses within a burst,
a third time interval between a positive and a negative pulse of at least one bipolar pulse,
a pulse width of a positive and/or negative pulse of at least one bipolar pulse,
a value of a pulse deflection of a positive and/or negative pulse of at least one bipolar pulse.

4. The device according to claim 1, wherein the transmitted measuring signal has a measuring signal level which is equal to a burst-signal sequence level.

5. A system for the irreversible electroporation of a tissue, comprising the device according to claim 1 and a monopolar catheter, wherein the catheter comprises a distal end and the electrode pair is configured as a first electrode and a body surface electrode, wherein the first electrode is arranged at the distal end of the catheter and the body surface electrode is arranged on a body surface of a patient.

6. A system for the irreversible electroporation of a tissue, comprising the device according to claim 1 and a bipolar catheter, wherein the catheter comprises a distal end and the electrode pair is arranged at the distal end.

7. A method for the irreversible electroporation of a tissue, comprising the steps of:
providing an electrical signal generator adapted to generate and transmit an electrical signal in accordance with a signal protocol to be received;
providing an electrode pair which is connected to the electrical signal generator and is adapted to receive the electrical signal and to close an electrical connection via the tissue lying between the electrode pair;
providing an evaluation and control unit connected to the signal generator;
in a first operating phase, transmitting a measuring signal protocol to the signal generator by means of the evaluation and control unit;
in the first operating phase, transmitting at least one measuring signal through the tissue by means of the signal generator and the electrode pair;
in the first operating phase, determining a tissue impedance on the basis of the measuring signal transmitted through the tissue;
in a second operating phase, adjusting at least one format of a burst-signal sequence protocol by means of the evaluation and control unit on the basis of the tissue impedance in order to specify an amount of energy per burst;
in a third operating phase, transmitting the adjusted burst-sequence protocol to the signal generator by means of the evaluation and control unit.

8. The method according to claim 7, wherein the formats of the burst-signal sequence protocol specify properties and the amount of energy per burst of a burst-signal sequence to be generated by the signal generator, comprising
a first number of bursts within the burst-signal sequence,
at least one first time interval between at least two successive bursts of the burst-signal sequence,
a second number of bipolar pulses within a burst,
at least one second time interval between at least two successive bipolar pulses within a burst,
a third time interval between a positive and a negative pulse of at least one bipolar pulse,
a pulse width of a positive and/or negative pulse of at least one bipolar pulse, and
a value of a pulse deflection of a positive and/or negative pulse of at least one bipolar pulse.
